(19) United States
(12) Patent Application Publication
(10) Pub. No.: US 2014/0024875 A1
(43) Pub. Date: Jan. 23, 2014

(54) PROCESS FOR TREATING AN OUTPUT FROM A HYDROCARBON CONVERSION WITH REMOVAL OF HYDROGEN HALIDES AND SUBSEQUENT WASH

(71) Applicant: BASF SE, Ludwigshafen (DE)

(72) Inventors: Katharina Spuhl, Forest (BE); Markus Schmitt, Heidelberg (DE); Jochen Bürkle, Mannheim (DE); Joni Joni, Sulzbach (DE); Steffen Tschirschwitz, Mannheim (DE); Daniel Pfeiffer, Neustadt (DE); Stefan Bitterlich, Dirmstein (DE); Michael Hübner, Lampertheim (DE); Pawel Czajka, Mannheim (DE)

(73) Assignee: BASF SE, Ludwigshafen (DE)

(21) Appl. No.: 13/937,436

(22) Filed: Jul. 9, 2013

Related U.S. Application Data
(60) Provisional application No. 61/670,135, filed on Jul. 11, 2012.

(51) Int. Cl. C07C 7/00

(52) U.S. Cl. CPC C07C 7/005
USPC 585/803

(57) ABSTRACT

The present invention relates to a process for treating an output from a hydrocarbon conversion, wherein the hydrocarbon conversion is performed in the presence of an acidic ionic liquid. The hydrocarbon conversion is preferably an isomerization. First of all, the hydrogen halide is drawn off in an apparatus from a mixture which originates from the hydrocarbon conversion and comprises at least one hydrocarbon and at least one hydrogen halide, and then the mixture depleted of hydrogen halide is subjected to a wash.

This patent application claims the benefit of pending U.S. provisional patent application Ser. No. 61/670,135 filed on Jul. 11, 2012, incorporated in its entirety herein by reference.

The present invention relates to a process for treating an output from a hydrocarbon conversion, wherein the hydrocarbon conversion is performed in the presence of an acidic ionic liquid. The hydrocarbon conversion is preferably an isomerization. First of all, the hydrogen halide is drawn off in an apparatus from a mixture which originates from the hydrocarbon conversion and comprises at least one hydrocarbon and at least one hydrogen halide, and then the mixture depleted of hydrogen halide is subjected to a wash.

Ionic liquids can be used in various hydrocarbon conversion processes; they are especially suitable as catalysts for the isomerization of hydrocarbons. A corresponding use of an ionic liquid is disclosed, for example, in WO 2011/069929, where a specific selection of ionic liquids is used in the presence of an olefin for isomerization of saturated hydrocarbons, more particularly for isomerization of methylcyclopentane (MCP) to cyclohexane. A similar process is described in WO 2011/069957, but the isomerization therein is not effected in the presence of an olefin, but with a copper(II) compound.

In addition to the ionic liquid, it is also possible to use hydrogen halides, preferably as cocatalysts, in hydrocarbon conversion processes, especially in isomerization processes. Frequently, the hydrogen halides are used in gaseous form. In order to be able to better utilize the cocatalytic effect of the hydrogen halides, a partial pressure of 1-10 bar of hydrogen halide, especially of hydrogen chloride, is generally established over the reaction mixture in which the isomerization is performed. However, a certain portion of the hydrogen halide used is dissolved in the hydrocarbons and consequently discharged from the isomerization reaction. This proportion of hydrogen halide dissolved in the hydrocarbons has to be removed again from the hydrocarbons after the isomerization, particularly due to the corrosive properties of the hydrogen halide, and this removal is in practice frequently associated with problems.

US-A 2011/0155632 discloses a process for preparing products with a low hydrogen halide content, wherein the content of hydrogen halides is reduced in at least two separation steps, by stripping or distillation from a mixture which originates from a reactor and comprises an ionic liquid as a catalyst. In one embodiment of the process described in US-A 2011/0155632, the ionic liquid used as a catalyst is recycled into an alkylation reactor from a downstream phase separator, and hydrogen chloride is recycled from a first distillation column downstream of the phase separator and an isobutane-comprising stream from a second distillation column further downstream into the alkylation reactor. US-A 2011/0155632, however, does not disclose anywhere that a hydrogen halide, especially hydrogen chloride, can be removed effectively in two process stages, the second process stage being a wash with an aqueous medium, from a product, for example from an alkylation product or an isomerization product. In contrast, in the execution variants described therein, the use of two separation stages without the use of an aqueous wash medium, more particularly of two distillation steps, is absolutely necessary in order to obtain a low content of hydrogen halide in the reaction product. A similar disclosure to that in US-A 2011/0155632 is present in US-A 2011/0155640, but the process described therein relates to a hydrocarbon conversion.

U.S. Pat. No. 3,271,467 discloses a process and a corresponding apparatus for maintaining the hydrogen halide concentration in a hydrocarbon conversion, wherein the catalyst used is a metal halide and the hydrogen halide is used as a promoter. Suitable metal halides are, for example, aluminum chloride, aluminum bromide, boron trifluoride or halides of zinc, tin, antimony or zirconium, but such compounds are not ionic liquids. The hydrocarbon conversion may, for example, be an isomerization of methylcyclopentane (MCP) to cyclohexane. In a (first) stripping apparatus, a stream rich in gaseous hydrogen halide is removed from the hydrocarbon-containing output from the hydrocarbon conversion and discharged from the arrangement. A second stream enriched in hydrogen halide is passed from the stripping apparatus into an absorption apparatus, in order to selectively remove the hydrogen halide present in this stream over a solid absorber therein. The hydrogen halide thus removed is removed again from the solid absorber and recycled into the system.

WO 2010/075038 discloses a process for reducing the content of organic halides in a reaction product, these being formed as a result of a hydrocarbon conversion process in the presence of a halogen-comprising catalyst based on an acidic ionic liquid. The hydrocarbon conversion process is especially an alkylation; this process can optionally also be performed as an isomerization. The organic halides are removed from the reaction product by washing with an aqueous alkaline solution. The use of hydrogen halide as a cocatalyst of ionic liquids in hydrocarbon conversions such as isomerization processes and the associated removal of hydrogen halide from the isomerization product, however, is not disclosed in WO 2010/075038.

It is an object of the present invention to provide a novel process for removing hydrogen halide from a mixture which is obtained in a hydrocarbon conversion, especially in an isomerization, of at least one hydrocarbon in the presence of an acidic ionic liquid.

The object is achieved by a process for treating an output from a hydrocarbon conversion, the hydrocarbon conversion being performed in the presence of an acidic ionic liquid having the composition K1AlₙX₍₃ₙ₊₁₎ where K1 is a monovalent cation, X is halogen and 1<n<2.5, the output comprises a mixture (G1) and mixture (G1) at least one hydrocarbon and at least one hydrogen halide (HX), which comprises the following steps:
a) feeding mixture (G1) into an apparatus (V1), and drawing off a mixture (G1b) comprising at least 50% by weight, preferably at least 70% by weight, of the hydrogen halide (HX) present in (G1) from (V1),
b) discharging a mixture (G2) from apparatus (V1), mixture (G2) comprising at least one hydrocarbon and an amount of at least one hydrogen halide (HX) reduced by mixture (G1b) compared to mixture (G1),
c) washing mixture (G2) with an aqueous medium to obtain a mixture (G3) comprising at least one hydrocarbon and not more than 100 ppm by weight, preferably not more than 10 ppm by weight, of hydrogen halide (HX) (based on the total weight of (G3)).

### BRIEF DESCRIPTION OF THE FIGURES

FIG. 1 illustrates a preferred embodiment according to the process according to the invention.

FIG. 2 illustrates example 1 schematically.

FIG. 3 illustrates comparative example 2 schematically.

By virtue of the process according to the invention, it is advantageously possible to remove hydrogen halide present/dissolved in the corresponding product (hydrocarbons) again after a hydrocarbon conversion, especially an isomerization, from this product again, especially from an isomerization product. Due to the optional, at least partial recycling of the hydrogen halide, this can additionally be reused in the process.

The processes described in the two applications US-A 2011/0155632 and US-A 2011/0155640 are disadvantageous particularly because the problem addressed therein, that of reducing the hydrogen halide content in products obtained in hydrocarbon conversion processes, is solved only by a purely distillative route. However, at least for hydrocarbons having up to 7 carbon atoms, the depletion of hydrogen halide (HX) down to a very low residual concentrations (<19 ppm by weight) requires execution of the distillation as a rectification in a column. This is because it is impossible to attain, by means of a simple vaporization, an HX concentration of a few ppm by weight in the low-HX fraction without discharging a majority of the hydrocarbon introduced into the vaporization with the HX-rich fraction, which would be associated with considerable process technology and economic disadvantages.

By virtue of significant concentration of the HX in the top product, rectification does enable more selective removal of the HX than one-stage vaporization, but is associated with a high level of expenditure because the jacket and internals of the rectifying column and the reboiler and top condenser thereof have to be designed such that they withstand the highly corrosive properties of the HX, which means that it is necessary to use costly specialty materials, this being disadvantageous especially given the complex geometries of the column internals, because the corrosion-resistant materials or coatings are either impossible here or are associated with high costs.

If the apparatus (V1) used in the process according to the invention is a flash apparatus or another simple vaporization, this is associated with the advantages which follow. The use of a flash apparatus or another simple vaporization in step a) is first of all less costly and simpler in apparatus terms, especially compared to the use of a rectifying column (due to the corrosiveness of the hydrogen halide, which is particularly disadvantageous given the complex geometries which exist in a column).

The use of a flash apparatus as apparatus (V1) is particularly advantageous since the separating effect in the flash apparatus is achieved merely by lowering the pressure with respect to the pressure selected for the hydrocarbon conversion, especially for the isomerization. Thus, no separate energy input is needed, and the corrosiveness of the hydrogen halide is less apparent as a result.

The above-described advantages of the process according to the invention become even more apparent if step a) of the invention is preceded by upstream connection of a phase separation unit, especially a phase separator, to the apparatus (V1), especially a flash apparatus.

The process according to the invention for treatment of an output from a hydrocarbon conversion, wherein the hydrocarbon conversion is performed in the presence of an acidic ionic liquid, is defined in detail hereinafter.

Hydrocarbon conversions as such are known to those skilled in the art. The hydrocarbon conversion is preferably selected from an alkylation, a polymerization, a dimerization, an oligomerization, an acylation, a metathesis, a polymerization or copolymerization, an isomerization, a carbonylation or combinations thereof.

Alkylations, isomerizations, polymerizations etc. are known to those skilled in the art. Especially preferably in the context of the present invention, the hydrocarbon conversion is an isomerization.

In the context of the present invention, the hydrocarbon conversion is effected in the presence of an acidic ionic liquid having the composition K1AlₙX₍₃ₙ₊₁₎ where K1 is a monovalent cation, X is halogen and 1<n<2.5. Such acidic ionic liquids are known to those skilled in the art; they are disclosed (alongside further ionic liquids), for example, in WO 2011/069929. For example, mixtures of two or more acidic ionic liquids may be used, preference being given to using one acidic ionic liquid.

K1 is preferably an unsubstituted or at least partly alkylated ammonium ion or a heterocyclic (monovalent) cation, especially a pyridinium ion, an imidazolium ion, a pyridazinium ion, a pyrazolium ion, an imidazolinium ion, a thiazolium ion, a triazolium ion, a pyrrolidinium ion, an imidazolidinium ion or a phosphonium ion. X is preferably chlorine or bromine.

The acidic ionic liquid more preferably comprises, as a cation, an at least partly alkylated ammonium ion or a heterocyclic cation and/or, as an anion, a chloroaluminate ion having the composition AlₙCl₍₃ₙ₊₁₎ where 1<n<2.5. The at least partly alkylated ammonium ion preferably comprises one, two or three alkyl radicals (each) having 1 to 10 carbon atoms. If two or three alkyl substituents are present with the corresponding ammonium ions, the respective chain length can be selected independently; preferably, all alkyl substituents have the same chain length. Particular preference is given to trialkylated ammonium ions having a chain length of 1 to 3 carbon atoms. The heterocyclic cation is preferably an imidazolium ion or a pyridinium ion.

The acidic ionic liquid especially preferably comprises, as a cation, an at least partly alkylated ammonium ion and, as an anion, a chloroaluminate ion having the composition AlₙCl₍₃ₙ₊₁₎ where 1<n<2.5. Examples of such particularly preferred acidic ionic liquids are trimethylammonium chloroaluminate and triethylammonium chloroaluminate.

The acidic ionic liquid used in the context of the present invention is preferably used as a catalyst in the hydrocarbon conversion, especially as an isomerization catalyst. In addition, in the context of the present invention, the hydrocarbon conversion is also effected in the presence of a hydrogen halide (HX), preference being given to using the hydrogen halide (HX) as a cocatalyst.

The hydrogen halides (HX) used may in principle be any conceivable hydrogen halides, for example hydrogen fluoride (HF), hydrogen chloride (HCl), hydrogen bromide (HBr) or hydrogen iodide (HI). The hydrogen halides can optionally also be used as a mixture, but preference is given in the context of the present invention to using only one hydrogen halide. Preference is given to using the hydrogen halide whose halide moiety is also present in the above-described acidic ionic liquid (at least partly) in the corresponding anion. The hydrogen halide (HX) is preferably hydrogen chloride (HCl) or hydrogen bromide (HBr). The hydrogen halide (HX) is more preferably hydrogen chloride (HCl).

In principle, it is possible in the context of the present invention to use any hydrocarbons, provided that at least one of the hydrocarbons used can be subjected in the presence of the above-described acidic ionic liquids to a hydrocarbon conversion, especially to an isomerization. On the basis of his or her general specialist knowledge, the person skilled in the art knows which hydrocarbons can be subjected by means of acidic ionic liquids to a hydrocarbon conversion, and more particularly which hydrocarbons are isomerizable. For example, it is possible to use mixtures of two or more hydrocarbons, but it is also possible to use only one hydrocarbon. Thus, it is possible in the context of the present invention that, in a mixture comprising two or more hydrocarbons, only one of these hydrocarbons is subjected to a hydrocarbon conversion, especially isomerized. Optionally, such mixtures may also comprise compounds which are not themselves hydrocarbons but are miscible therewith.

The hydrocarbon used in the hydrocarbon conversion is preferably methylcyclopentane (MCP) or a mixture of methylcyclopentane (MCP) with at least one further hydrocarbon selected from cyclohexane, n-hexane, isohexanes, n-heptane, isoheptanes, methylcyclohexane or dimethylcyclopentanes.

More preferably, a mixture of methylcyclopentane (MCP) with at least one further hydrocarbon selected from cyclohexane, n-hexane, isohexanes, n-heptane, isoheptanes, methylcyclohexane or dimethylcyclopentanes is used.

The hydrocarbon conversion can in principle be performed in all apparatuses known for such a purpose to the person skilled in the art. The corresponding apparatus is preferably a stirred tank or a stirred tank cascade. A “stirred tank cascade” means that two or more, for example three or four, stirred tanks are connected in succession (in series).

As already explained above, due to the hydrocarbon conversion in the presence of an acidic ionic liquid and of a hydrogen halide (HX), the chemical structure of at least one of the hydrocarbons used is altered. The hydrocarbons obtained in the hydrocarbon conversion are present in a mixture (G1). Mixture (G1) thus differs in terms of (chemical) composition and/or amount of the hydrocarbons present therein from the corresponding hydrocarbon composition present prior to the hydrocarbon conversion, especially prior to the isomerization. Since the hydrocarbon conversion to be performed in such hydrocarbon conversions, especially in isomerization processes, frequently does not proceed to an extent of 100% (i.e. to completion), the product generally still also comprises the hydrocarbon with which the hydrocarbon conversion has been performed (in a smaller amount than before the isomerization). If, for example, MCP is to be isomerized to cyclohexane, the isomerization product frequently comprises a mixture of cyclohexane and (in a smaller amount than before the isomerization) MCP.

As well as the hydrocarbons, mixture (G1) preferably comprises at least one hydrogen halide (HX) and optionally further components. The hydrogen halide (HX) present in mixture (G1) is generally the same hydrogen halide as that used in the hydrocarbon conversion (preferably as a cocatalyst), because the chemical structure of the hydrogen halide is not normally altered by the hydrocarbon conversion, but there may be partial exchange of the anionic moiety of the hydrogen halide used with other halide ions present in the process. As a further component, mixture (G1) preferably comprises the above-described ionic liquid. Mixture (G1) comprises between 10 and 99% by weight, preferably between 50 and 95% by weight, of acidic ionic liquid (the stated amounts are based on the total weight of hydrocarbons and hydrogen halide in mixture (G1)).

The hydrocarbon present in mixture (G1)—i.e. as the product of the hydrocarbon conversion—is preferably cyclohexane. The hydrocarbon present in mixture (G1) is more preferably cyclohexane or a mixture of cyclohexane with at least one further hydrocarbon selected from methylcyclopentane (MCP), n-hexane, isohexanes, n-heptane, isoheptanes, methylcyclohexane and dimethylcyclopentanes.

The hydrocarbon present in mixture (G1) is especially preferably a mixture of cyclohexane, MCP and at least one further hydrocarbon. The further hydrocarbon is preferably selected from n-hexane, isohexanes, n-heptane, isoheptanes, methylcyclohexane or dimethylcyclopentanes. If the hydrocarbon conversion performed is an isomerization, the proportion of branched hydrocarbons in mixture (G1) is preferably less than 10% by weight (based on the sum of all hydrocarbons present in mixture (G1)).

In a preferred embodiment of the present invention, mixture (G1) comprises i) as a hydrocarbon a mixture of cyclohexane with at least one further hydrocarbon selected from methylcyclopentane (MCP), n-hexane, isohexanes, n-heptane, isoheptanes, methylcyclohexane and dimethylcyclopentanes, ii) hydrogen chloride (HCl) and iii) an acidic ionic liquid which has, as a cation, an at least partly alkylated ammonium ion and, as an anion, a chloroaluminate ion having the composition AlₙCl₍₃ₙ₊₁₎ where 1<n<2.5.

In a further preferred embodiment of the present invention, at least 80% by weight of the hydrocarbons present in mixture (G1) have at least 5 carbon atoms per molecule. These hydrocarbons especially preferably have at least 6 carbon atoms per molecule.

Mixture (G1) is at first present in the apparatus in which the hydrocarbon conversion is performed. In the context of the process according to the invention, mixture (G1) is discharged from this apparatus as the output. In other words, this means that the output comprises mixture (G1) and the output or mixture (G1), after it has left the apparatus for performance of the hydrocarbon conversion, is subjected to the steps a) to c) of the invention (defined below in the text).

If the hydrocarbon conversion in the context of the present invention is an isomerization, the isomerization is preferably performed as follows. The performance of an isomerization of hydrocarbons in the presence of an ionic liquid as a catalyst and a hydrogen halide as a cocatalyst is known to those skilled in the art. The hydrocarbons and the ionic liquid in the isomerization preferably each form a separate phase, though portions of the ionic liquid may be present in the hydrocarbon phase and portions of the hydrocarbons in the ionic liquid phase. The hydrogen halide, especially hydrogen chloride, is introduced, preferably in gaseous form, into the apparatus for performance of the isomerization. The hydrogen halide may be present, at least in portions, in the two aforementioned liquid phases; the hydrogen halide preferably forms a separate, gaseous phase.

The isomerization is preferably performed at a temperature between 0° C. and 100° C., especially preferably at a temperature between 30° C. and 60° C. It is additionally preferred that the pressure in the isomerization is between 1 and 20 bar abs. (absolute), preferably between 2 and 10 bar abs.

The isomerization is preferably performed in the apparatus in such a way that two liquid phases and one gas phase are present in a stirred tank or a stirred tank cascade. The first liquid phase comprises the acidic ionic liquid to an extent of at least 90% by weight and the second liquid phase comprises the hydrocarbons to an extent of at least 90% by weight. The gas phase comprises at least one hydrogen halide, preferably hydrogen chloride, to an extent of at least 90% by weight. Optionally, a solid phase may also be present, this comprising components in solid form from which the ionic liquid is formed, for example AlCl₃. The pressure and composition of the gas phase are set here such that the partial pressure of the gaseous hydrogen halide, especially of HCl gas, in the gas phase is between 1 and 20 bar abs., preferably between 2 and 10 bar abs.

In step a) of the process according to the invention, mixture (GI) is fed into an apparatus (V1), and a mixture (G1b) comprising at least 50% by weight of the hydrogen halide (HX) present in (G1) is drawn off from (V1). Mixture (G1b) is preferably drawn off from apparatus (VI) partly or fully in gaseous form, more preferably fully in gaseous form. Preferably at least 50%, more preferably at least 70% of the hydrogen halide introduced with mixture (G1) is drawn off with mixture (G1b).

Preference is given to partial or full recycling of mixture (G1b) into the apparatus for performance of the hydrocarbon conversion, optionally increasing the pressure by means of a suitable apparatus, for example a jet compressor, piston compressor, turbo compressor or screw compressor. Mixture (G1b) is preferably recycled fully into the apparatus for performance of the hydrocarbon conversion. If complete recycling of mixture (G1b) is not performed, the excess amounts of mixture (G1b) can be discharged from the process according to the invention and discarded or sent to a further process step.

The apparatus (V1) used to perform the gaseous drawing-off (removal) of the hydrogen halide (HX) from mixture (G1) may in principle be any apparatus known for such a purpose to the person skilled in the art, preferably a concentration apparatus, a rectifying column, an apparatus for flash vaporization (flash apparatus) or a stripping apparatus. V1 is especially preferably a flash apparatus. Apparatus (V1) is intended, in the context of the process according to the invention, preferably to perform removal of the hydrogen halides from the hydrocarbons.

In the context of the present invention, step a) should be understood such that, in the case of use of a flash apparatus as apparatus (V1), an appropriate flash operation (flashing) is performed with mixture (G1). The same applies to the further configurations of apparatus (V1) detailed above, such as stripping apparatus or rectifying column.

In the context of the present invention, the term “concentration”, which is performed in a corresponding concentration apparatus, is understood to mean the following: a characteristic feature of concentration is that a portion of the liquid mixture to be separated is vaporized with supply of heat and is condensed after removal from the remaining liquid mixture. For the original liquid phase, a vapor phase is thus produced, in which the relatively low-boiling mixture components are enriched.

In the context of the present invention, the term “rectification”, which is performed in a corresponding rectifying column (rectifying apparatus), also called rectification column or rectification apparatus, is understood to mean the following: in rectification, the vapor produced by distillation is conducted in countercurrent to a portion of the condensate thereof in a rectifying column. In this way, more volatile components are enriched in the top product and less volatile components in the bottom product of the rectifying column.

In the context of the present invention, the term “flashing”, which is performed in a corresponding flash apparatus and can also be referred to as flash vaporization, is understood to mean the following: Flash vaporization (flashing) involves decompressing a liquid mixture into a suitable apparatus (flash apparatus), for example into a vapor/liquid separation vessel (i.e., in a suitable apparatus, for example a valve, a lowering of the pressure takes place, this being sufficient to cause a portion of the liquid mixture to vaporize spontaneously). The liquid mixture may originate, for example, from a reaction stage operated at higher pressure. However, it is also possible to effect preheating in a preheater, for example to boiling temperature, in which case the pressure in the preheater must be higher than the pressure in the downstream separation vessel. The vapor forming in the course of decompression has a higher proportion of relatively low-boiling components than the mixture entering the separator. The flash vaporization thus ensures partial separation of the incoming mixture, in which case the separator can act as a sole theoretical plate. The flashing can also be combined with heat supply to the liquid mixture which remains in the flashing operation, for example by means of a circulation vaporizer connected to the separation vessel.

In the context of the present invention, the term “stripping”, which is performed in a corresponding stripping apparatus, is understood to mean the following: in the course of stripping, one or more relatively low-boiling components are depleted from a liquid, these being contacted, preferably in a countercurrent column, with gases such as nitrogen, air or steam, such that the decrease in the partial pressure of the relatively low-boiling components in the gas phase brought about by the gas results in a decrease in the solubility thereof in the liquid.

Further information regarding the above terms “distillation”, “rectification”, “vaporization”, “flashing” and/or “stripping” can be found in the following textbooks: Sattler, Thermische Trennverfahren [Thermal Separation Processes], VCH, 1988; Perry's Chemical Engineers' Handbook, 7th edition; R. H. Perry, D. W. Green, 1997, McGraw-Hill.

In step b) of the process according to the invention, a mixture (G2) is discharged from apparatus (V1), mixture (G2) comprising at least one hydrocarbon and an amount of at least one hydrogen halide (HX) reduced by mixture (G1b) compared to mixture (G1).

For example, mixture (G2) may comprise an amount of at least one hydrogen halide (HX) reduced by at least 50%. Preferably, mixture (G2) comprises an amount of at least one hydrogen halide (HX) reduced compared to mixture (G1) by at least 70%.

In step c) of the process according to the invention, mixture (G2) is washed with an aqueous medium to obtain a mixture (G3) comprising at least one hydrocarbon and not more than 100 ppm by weight, preferably not more than 10 ppm by weight, of hydrogen halide (HX) (based on the total weight of (G3)).

In the context of the process according to the invention, it is preferable that mixture (G3) obtained in process step c) (wash step c), with regard to the composition and/or amount of the hydrocarbons present therein, corresponds completely or at least substantially to mixtures (G1) and (G2). The expression “corresponds substantially” shall be understood in this context to mean that at least 90% by weight, preferably at least 95% by weight, especially at least 99% by weight, of the amount of hydrocarbons present in mixture (G1) is also present in mixture (G3). Especially preferably, mixture (G3) does not comprise any further components apart from at least one hydrocarbon and not more than 100 ppm by weight, preferably not more than 10 ppm by weight of hydrogen halide.

Preference is given to performing process step c) in such a way that the wash according to step c) comprises at least two wash steps:
c1) in a first wash step, the aqueous medium used has a pH >9, preferably >12,
c2) in a second wash step, the aqueous medium used has a pH between 5 and 9, preferably between 6 and 8.

The aqueous medium in the first wash step preferably comprises an alkali metal hydroxide, especially preferably NaOH. The aqueous medium in the second wash step is preferably water, especially preferably demineralized water.

Optionally, process step c) can be performed in such a way that step c2) can be performed prior to step c1). In this process variant, washing is thus effected first with an aqueous medium of relatively low pH, followed by the wash with an aqueous medium of higher pH. In addition, it is also possible to perform several steps c1) and several steps c2) in succession, optionally in alternating sequence.

Preference is given in the context of the present invention to performing wash step c) in two stages, first step c1) and then step c2).

In one embodiment of the present invention, only a one-stage wash step c) is performed, in which case the aqueous medium has a pH of 5 to 9, preferably between 6 and 8, and is especially preferably demineralized water. In this embodiment, wash step c) is preferably performed in an extraction column operated in countercurrent or a mixer-settler arrangement.

In addition, it is preferable in the context of the present invention that step c) is performed using at least one dispersion and phase separation unit or at least one extraction column per wash stage. The dispersion and phase separation unit is preferably a mixer-settler apparatus (combination of a stirred tank with a downstream phase separator), a combination of static mixers with phase separator or a combination of mixing pump with phase separator.

It is additionally preferred in the context of the present invention that, in a multistage, especially two-stage, wash, mixture (G2) is conducted in countercurrent to the aqueous medium. It is especially preferred in the context of the present invention that mixture (G2) discharged from the apparatus (V1) is washed with the aqueous medium (according to step c)) without any intermediate steps.

In another embodiment, the wash step c) is performed in a multistage mixer-settler apparatus, preferably operated in countercurrent, or extraction is effected with water in an extraction column operated in countercurrent.

In the case of the mixer-settler apparatus or extraction column, a further wash stage is preferably connected downstream thereof in flow direction of the mixture (G2) (comprising the hydrocarbons), this being fed with fresh water. In the aqueous outlet thereof is an apparatus for continuous measurement of the pH or the electrical conductivity, in order thus to monitor the complete removal of the non-hydrocarbon components, especially HCl.

Preference is given to performing steps a) and b) of the invention according to at least one, more preferably according to all three, of the following variants i) to iii):
i) at least 95% by weight of mixture (G2) discharged from apparatus (V1) is liquid (in step b)),
ii) at least 95% by weight of mixture (G1b) drawn off from apparatus (V1) is gaseous (in step a)),
iii) the discharged mixture (G2) is at most 150 K, preferably at most 100 K, hotter than the mixture (G1b) drawn off (in step b)).

In a particularly preferred embodiment of the present invention, a one-stage vaporization, especially a one-stage flash vaporization, takes place in apparatus (V1) and the mixture (G2) discharged from apparatus (V1) is washed with the aqueous medium without any intermediate steps.

FIG. 1 once again illustrates the process according to the invention in a preferred embodiment. In this embodiment, at least partial recycling of the mixture (G1b) removed in step a) of the invention, preferably in gaseous form, into the apparatus (V) is also performed. R1 represents the apparatus in which the hydrocarbon conversion, especially an isomerization, is performed. This is preferably a stirred tank or a stirred tank cascade. Apparatus (V1) is preferably a vaporizer, especially a flash apparatus. The arrow coming from apparatus (V1) and pointing upward shows that it is optionally also possible in the context of the process according to the invention to perform only partial recycling of the mixture (G1b) removed in step a), or that mixture (G1b) is supplied to a further process step, preferably to a material separation. This is especially advantageous when (V1) is performed as a stripping operation. In this case, full or partial separation of the hydrogen halide from the stripping gas used can be performed in the process step, and the hydrogen halide-enriched stream thus obtained can be recycled fully or partly into the apparatus for performance of the hydrocarbon conversion. According to FIG. 1, the mixture (G2) discharged from apparatus (V1) is washed with the aqueous medium (according to step c)) without any intermediate steps. In FIG. 1, the inventive wash step c) is referred to in simplified form with the abbreviation “W”. The wash step c) according to FIG. 1 may, as described above, be performed in one or more stages, in which case preference is given to performing a multistage, especially two-stage, wash of mixture (G2) in countercurrent to the aqueous medium, and/or a dispersion and phase separation unit, especially a mixer-settler apparatus, is used.

In a further preferred embodiment of the present invention, mixture (G1) is discharged as an output from the apparatus in which the hydrocarbon conversion is performed, conducted through a phase separation unit, especially into a phase separator, and then fed into the apparatus (V1). In other words, this means that an intermediate step is performed after the performance of the hydrocarbon conversion and prior to performance of step a) of the invention. In this intermediate step, the acidic ionic liquid present in mixture (G1) is preferably fully or at least partly removed from mixture (G1), and then mixture (G1) depleted of acidic ionic liquid is fed into the apparatus (V1).

Preferably at least 90%, more preferably at least 99%, of the acidic ionic liquid is removed from mixture (G1) in the phase separation unit and optionally recycled into the apparatus in which the hydrocarbon conversion is performed. Especially preferably, the acidic ionic liquid removed from mixture (G1) in the phase separation unit is recycled fully or partly into the apparatus for performance of the hydrocarbon conversion, especially for performance of an isomerization.

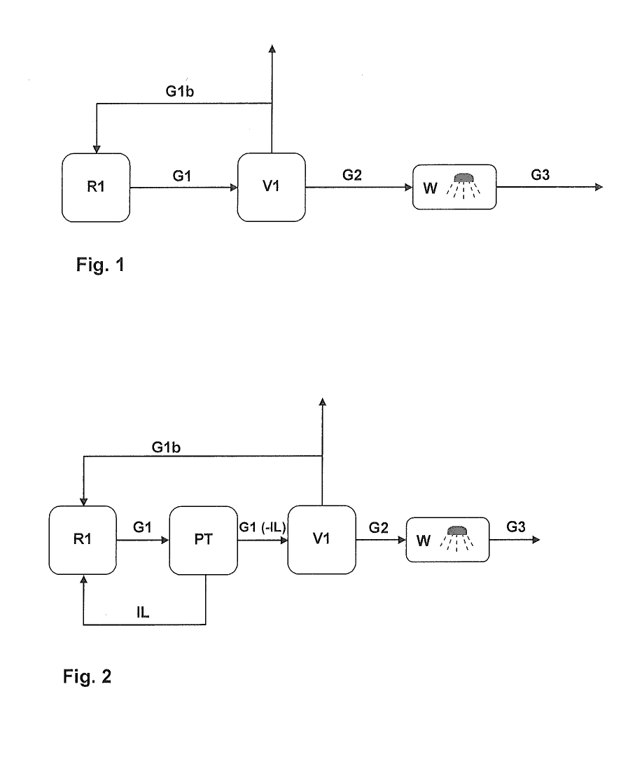

The above-described further preferred embodiment of the present invention is additionally illustrated in FIG. 2. In FIG. 2, the abbreviations, arrows and other symbols have similar meanings to those explained above for FIG. 1; PT means phase separation unit, IL means acidic ionic liquid.

In the context of the present invention, cyclohexane is preferably isolated from mixture (G3). Processes and apparatuses for removal of cyclohexane from mixture (G3) are known to those skilled in the art.

The present invention is to be illustrated hereinafter by examples.

For the simulation calculation, BASF's own software Chemasim was used (in the case of use of the commercially available software Aspen Plus (manufacturer: AspenTech, Burlington, Mass., USA), the same results would be obtained). The following substances or mixtures are used for the example calculation:

a) Hydrocarbon mixture (A) having the composition

| | |
|---|---|
| 2-methylpentane | 0.66% by wt. |
| 3-methylpentane | 1.95% by wt. |
| n-hexane | 28.85% by wt. |
| methylcyclopentane | 51.02% by wt. |
| cyclohexane | 17.44% by wt. |
| 2,3-dimethylbutane | 0.05% by wt. |
| other hydrocarbons | 0.03% by wt. |

b) Hydrogen chloride gas (B)

c) Ionic liquid (IL), specifically trimethylammonium heptachlorodialuminate (TMA-IL).

For the examples described, (A) and (B) are mixed such that the resulting mixture after phase separation G1(-IL) has an HCl content of 1.5% by weight.

### 1. EXAMPLE WITH HCl REMOVAL OR RECYCLING BY MEANS OF A FLASH APPARATUS (V1) AND SUBSEQUENT WASHING WITH AN AQUEOUS MEDIUM

Example 1 is performed according to the embodiment shown schematically in FIG. 2.

In an apparatus R1, isomerization of a hydrocarbon mixture (A) takes place in the presence of an ionic liquid (trimethylammonium heptachlorodialuminate—TMA-IL), which serves as the catalyst. This isomerization relates preferably to the conversion of methylcyclopentane to cyclohexane. The volume ratio of ionic liquid to organic phase is 5. Additionally supplied are hydrogen chloride gas (B) for stabilization of the IL, and the recycle streams G1b from a flash apparatus V1, and IL from a phase separation PT. For apparatus R1, an operating pressure of 3.5 bar (abs) and a temperature of 50° C. are assumed. The resulting mixture G1 is passed into an apparatus for phase separation PT. In a simplification, it is assumed for this apparatus that the TMA-IL is removed and recycled fully and as a pure IL phase. The organic phase G1(-IL) is passed into the flash apparatus V1, where it is decompressed to an operating pressure of 1 bar (abs). The resulting gas component G1b is recycled into the apparatus R1. The liquid mixture G2 is discharged from V1, and the organic stream G2 is depleted further of hydrogen chloride in a subsequent step W by washing (multistage) with an aqueous medium.

The calculated properties and compositions of the streams are shown in table 1.

TABLE 1
Properties and composition of the streams from example 1

| Stream: | | | A | IL | B | G1 | IL | G1-IL | G1b | G2 | G3 |
|---|---|---|---|---|---|---|---|---|---|---|---|
| From apparatus | | | | PT | | V1 | PT | PT | V2 | V2 | W |
| To apparatus | | | V1 | V1 | V1 | PT | V1 | V2 | V1 | W | |
| Phase | | | fluid | fluid | gas | fluid | fluid | fluid | gas | fluid | fluid |
| | | | Curr. | Curr. | Curr. | Curr. | Curr. | Curr. | Curr. | Curr. | Curr. |
| Properties | | Unit | value | value | value | value | value | value | value | value | value |
| Temperature | | ° C. | 45.00 | 50.00 | 50.00 | 50.00 | 50.00 | 50.00 | 45.40 | 45.40 | 33.93 |
| Pressure | | bar | 3.50 | 3.50 | 3.50 | 3.50 | 3.50 | 3.50 | 1.00 | 1.00 | 1.00 |
| Enthalpy | | kW | 0.124 | 1.369 | 0.002 | 1.511 | 1.369 | 0.142 | 0.017 | 0.124 | 0.091 |
| Mean molar mass | | kg/kmol | 84.79 | 362.25 | 36.46 | 274.96 | 362.25 | 83.14 | 55.09 | 84.41 | 84.75 |
| Thermal conductivity | | W/(m*K) | 0.114007 | 0.150 | 0.015 | 0.143 | 0.150 | 0.112 | 0.014 | 0.114 | 0.117 |
| Viscosity eta | | mPa*s | 0.368 | 21.012 | 0.016 | 6.067 | 21.012 | 0.396 | 0.011 | 0.442 | 0.519 |
| Surface tension | | N/m | 0.018 | 0.013 | | 0.014 | 0.013 | 0.018 | | 0.019 | 0.021 |
| Specific heat | | kJ/kg/K | 2.09 | 2.00 | 0.80 | 2.01 | 2.00 | 2.10 | 1.22 | 2.07 | 2.02 |
| Density | | kg/m³ | 700.37 | 1385.51 | 4.75 | 1268.90 | 1385.51 | 702.65 | 2.08 | 709.59 | 720.69 |
| Mass flow rate | | kg/h | 4.983 | 49.287 | 0.017 | 54.435 | 49.287 | 5.147 | 0.147 | 5.000 | 4.983 |
| | | | Curr. | Curr. | Curr. | Curr. | Curr. | Curr. | Curr. | Curr. | Curr. |
| Concentrations | Mol. M. | Unit | value | value | value | value | value | value | value | value | value |
| 2-M-pentane | 86.179 | g/g | 0.0066 | | | 0.0043 | | 0.0457 | 0.0429 | 0.0458 | 0.0459 |
| 3-M-pentane | 86.179 | g/g | 0.0195 | | | 0.0039 | | 0.0408 | 0.0348 | 0.0410 | 0.0412 |
| Hexane | 86.179 | g/g | 0.2885 | | | 0.0197 | | 0.2084 | 0.1538 | 0.2100 | 0.2107 |
| M-CY-pentane | 84.163 | g/g | 0.5102 | | | 0.0114 | | 0.1210 | 0.0765 | 0.1223 | 0.1227 |
| Cyclohexane | 84.163 | g/g | 0.1744 | | | 0.0521 | | 0.5514 | 0.2646 | 0.5599 | 0.5617 |
| 2,2-DM-butane | 86.179 | g/g | 0.0000 | | | 0.0003 | | 0.0037 | 0.0048 | 0.0036 | 0.0036 |
| 2,3-DM-butane | 86.179 | g/g | 0.0005 | | | 0.0013 | | 0.0137 | 0.0147 | 0.0137 | 0.0137 |
| Water | 18.015 | g/g | | | | | | | | | 0.0001 |
| TMA-IL | 362.2511 | g/g | | 1.0000 | | 0.9054 | 1.0000 | | | | |
| HCL | 36.461 | g/g | | | 1.0000 | 0.0014 | | 0.0150 | 0.4079 | 0.0034 | 0.0000 |

### 2. COMPARATIVE EXAMPLE WITH DISTILLATIVE HCl REMOVAL

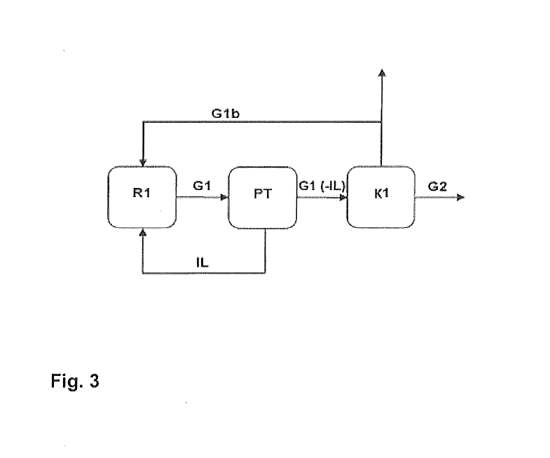

Comparative example 2 is shown schematically in FIG. 3. In contrast to example 1, however, no wash step c) is conducted, but a distillation column (K1) is used in place of the apparatus (V1). In an apparatus R1, an isomerization of a hydrocarbon mixture (A) takes place in the presence of an ionic liquid (trimethylammonium chloroaluminate—TMA-IL), which serves as the catalyst. This isomerization preferably relates to the conversion of methylcyclopentane to cyclohexane. The volume ratio of ionic liquid to organic phase is 5 Ill. In addition, hydrogen chloride gas (B) is supplied for stabilization of the IL. For apparatus V1, an operating pressure of 3.5 bar (abs) and a temperature of 50° C. are assumed. The resulting mixture G1 is passed into an apparatus for phase separation PT. In a simplification, it is assumed for this apparatus that the TMA-IL is removed and recycled completely and as a pure IL phase. The resulting organics G1(-IL) are passed into a distillation column K1. The hydrogen chloride present in G1 is removed by distillation therein, and the residual content of hydrogen chloride in the organic bottoms discharge should be less than 10 ppm by weight. The gaseous distillate is recycled into the apparatus R1.

The calculated properties and compositions of the streams are shown in table 2.

Table 2: Properties and composition of the streams from comparative example 2

TABLE 2
Properties and composition of the streams from comparative example 2

| Stream: | | | A | B | G1b | G1-IL | G1 IL PT | IL | G2 |
|---|---|---|---|---|---|---|---|---|---|
| From apparatus | | | | | V2 | V1 | PT | PT | V2 |
| To apparatus | | | V1 | V1 | V1 | PT | V2 | V1 | |
| Phase | | | fluid | gas | gas | fluid | fluid | fluid | fluid |
| Properties | | Unit | Curr. value | Curr. value | Curr. value | Curr. value | Curr. value | Curr. value | Curr. value |
| Temperature | | ° C. | 45.00 | 50.00 | 46.07 | 50.00 | 50.00 | 50.00 | 73.46 |
| Pressure | | bar | 3.50 | 3.50 | 1.00 | 3.50 | 3.50 | 3.50 | 1.00 |
| Enthalpy | | kW | 0.124 | 0.000 | 0.023 | 1.512 | 0.143 | 1.369 | 0.207 |
| Mean molar mass | | kg/kmol | 84.79 | 36.46 | 55.58 | 274.61 | 83.14 | 362.25 | 84.79 |
| Thermal conductivity | | W/(m*K) | 0.114 | 0.015 | 0.014 | 0.143 | 0.112 | 0.150 | 0.106 |
| Viscosity eta | | mPa*s | 0.368 | 0.016 | 0.010 | 6.035 | 0.395 | 21.012 | 0.330 |
| Surface tension | | N/m | 0.018 | | | 0.014 | 0.018 | 0.013 | 0.016 |
| Specific heat | | kJ/kg/K | 177.19 | 0.80 | 1.23 | 2.01 | 2.10 | 2.00 | 2.21 |
| Density | | kg/m³ | 700.37 | 4.75 | 2.09 | 1268.30 | 702.57 | 1385.51 | 682.16 |
| Mass flow rate | | kg/h | 4.983 | 0.000 | 0.195 | 54.466 | 5.178 | 49.287 | 4.983 |
| Concentrations | Mol. M. | Unit | Curr. value | Curr. value | Curr. value | Curr. value | Curr. value | Curr. value | Curr. value |
| 2-M-pentane | 86.179 | g/g | 0.0066 | | 0.0439 | 0.0044 | 0.0460 | | 0.0461 |
| 3-M-pentane | 86.179 | g/g | 0.0195 | | 0.0355 | 0.0039 | 0.0410 | | 0.0413 |
| Hexane | 86.179 | g/g | 0.2885 | | 0.1564 | 0.0198 | 0.2084 | | 0.2104 |
| M-Cy-pentane | 84.163 | g/g | 0.5102 | | 0.0777 | 0.0115 | 0.1209 | | 0.1226 |
| Cyclohexane | 84.163 | g/g | 0.1744 | | 0.2687 | 0.0524 | 0.5509 | | 0.5619 |
| 2,2-DM-butane | 86.179 | g/g | 0.0000 | | 0.0049 | 0.0004 | 0.0037 | | 0.0036 |
| 2,3-DM-butane | 86.179 | g/g | 0.0005 | | 0.0151 | 0.0013 | 0.0138 | | 0.0138 |
| Water | 18.015 | g/g | | | | | | | |
| TMA-IL | 362.2511 | g/g | | | | 0.9049 | | 1.0000 | |
| HCL | 36.461 | g/g | | 1.0000 | 0.3977 | 0.0150 | 0.0150 | | 0.0000 |

For K1, the number of plates used is 9. The heating output for the vaporizer her is 0.88 kW.

### 3. RESULTS OF THE SIMULATION CALCULATION

The results show that the relatively inexpensive distillative removal (heat requirement for the vaporizer and specific material demands for the column, since conditions are corrosive) of the hydrogen chloride from the organics (i.e. the hydrocarbons) can be replaced by a simple vaporization in a flash apparatus with downstream washing with a neutral medium. In this context, the desired reduction in the hydrogen chloride level in the organics can be kept below 10 ppm by weight.

The use of a flash apparatus is less expensive and simpler in apparatus terms compared with the distillative process, since it is less prone to corrosion because of non-complex geometries and, moreover, no separate energy input is needed.

1-20. (canceled)

21. A process for treating an output from a hydrocarbon conversion, the hydrocarbon conversion being performed in the presence of an acidic ionic liquid having the composition K1AlₓX₍₃ₙ₊₁₎ where K1 is a monovalent cation, X is halogen and 1<n<2.5, the output comprises a mixture (G1) and mixture (G1) at least one hydrocarbon and at least one hydrogen halide (HX), which comprises the following steps:
a) feeding mixture (G1) into an apparatus (V1), and drawing off a mixture (G1b) comprising at least 50% by weight of the hydrogen halide (HX) present in (G1) from (V1),
b) discharging a mixture (G2) from apparatus (V 1), mixture (G2) comprising at least one hydrocarbon and an amount of at least one hydrogen halide (HX) reduced by mixture (G1b) compared to mixture (G1),
c) washing mixture (G2) with an aqueous medium to obtain a mixture (G3) comprising at least one hydrocarbon and not more than 100 ppm by weight of hydrogen halide (HX) (based on the total weight of (G3)).

22. The process according to claim 21, wherein in step (a) the mixture (G1b) comprises at least 70% by weight of the hydrogen halide (HX) or in step c) the mixture (G3) comprises not more than 10 ppm by weight of hydrogen halide (HX).

23. The process according to claim 21, wherein at least 80% by weight of the hydrocarbons present in mixture (G1) have at least 5 carbon atoms per molecule.

24. The process according to claim 21, wherein the hydrocarbon conversion is selected from an alkylation, a polymerization, a dimerization, an oligomerization, an acylation, a metathesis, a polymerization or copolymerization, an isomerization, a carbonylation or combinations thereof.

25. The process according to claim 24, wherein the hydrocarbon conversion is an isomerization of methylcyclopentane (MCP) to cyclohexane.

26. The process according to claim 21, wherein the apparatus (V1) is a concentration apparatus, a rectifying column, a flash apparatus or a stripping apparatus.

27. The process according to claim 26, wherein the apparatus (V1) is a flash apparatus.

28. The process according to claim 21, wherein
i) at least 95% by weight of mixture (G2) discharged from apparatus (V1) is liquid,
ii) at least 95% by weight of mixture (G1b) drawn off from apparatus (V1) is gaseous and/or
iii) the discharged mixture (G2) is at most 150 K hotter than the mixture (G1b) drawn off.

29. The process according to claim 21, wherein the hydrogen halide (HX) is hydrogen chloride (HCl).

30. The process according to claim 27, wherein the wash according to step c) comprises at least two wash steps:
c1) in a first wash step, the aqueous medium used has a pH>9,
c2) in a second wash step, the aqueous medium used has a pH between 5 and 9, and optionally to perform step c2) prior to step c1).

31. The process according to claim 30, wherein step c1) the aqueous medium comprises NaOH or in step c2) the aqueous medium is demineralized water.

32. The process according to claim 21, wherein the aqueous medium has a pH of 5 to 9.

33. The process according to claim 21, wherein a one-stage vaporization takes place in apparatus (V1) and the mixture (G2) discharged from apparatus (V1) is washed with the aqueous medium without any intermediate steps.

34. The process according to claim 33, wherein a one-stage vaporization is a one-stage flash vaporization.

35. The process according to claim 21, wherein hydrogen halide (HX) drawn off via mixture (G1b) is recycled in step a) fully or partly into the apparatus in which the hydrocarbon conversion is performed.

36. The process according to claim 21, wherein mixture (G1) comprises, as the hydrocarbon, cyclohexane or a mixture of cyclohexane with at least one further hydrocarbon selected from methylcyclopentane (MCP), n-hexane, isohexanes, n-heptane, isoheptanes, methylcyclohexane or dimethylcyclopentanes.

37. The process according to claim 21, wherein the acidic ionic liquid comprises, as a cation, an at least partly alkylated ammonium ion or a heterocyclic cation and/or, as an anion, a chloroaluminate ion having the composition AlₙCl₍₃ₙ₊₁₎ where 1<n<2.5.

38. The process according to claim 21, wherein mixture (G1) additionally comprises between 10 and 99% by weight of acidic ionic liquid.

39. The process according to claim 21, wherein mixture (G1) is discharged as an output from the apparatus in which the hydrocarbon conversion is performed, conducted through a phase separation unit and then fed into the apparatus (V1).

40. The process according to claim 39, wherein the phase separation unit is a phase separator.

41. The process according to claim 39, wherein at least 90% of the acidic ionic liquid is removed from mixture (G1) in the phase separation unit and optionally recycled into the apparatus in which the hydrocarbon conversion is performed.

42. The process according to claim 21, wherein step c) is performed as a one-stage or multistage wash using at least one dispersion and phase separation unit or at least one extraction column per wash stage.

43. The process according to claim 42, wherein the dispersion and phase separation unit is a mixer-settler apparatus, a combination of static mixers with phase separator or a combination of mixing pump with phase separator.

44. The process according to claim 42, wherein, in a multistage wash, mixture (G2) is conducted in countercurrent to the aqueous medium.

45. The process according to claim 21, wherein cyclohexane is isolated from mixture (G3).
